(19) United States
(12) Patent Application Publication
(10) Pub. No.: US 2025/0108174 A1
(43) Pub. Date: Apr. 3, 2025

(54) Closed System Transfer Device Injection System

(71) Applicant: Becton, Dickinson and Company, Franklin Lakes, NJ (US)

(72) Inventors: Peter Abdelmessih, Oakland, NJ (US); Noel Antonio Medrano, Raleigh, NC (US); Alban Yee, Waldwick, NJ (US); Frank Lee, Jersey City, NJ (US); Rafael Gonzalez Macias, San Diego, CA (US)

(21) Appl. No.: 18/477,948

(22) Filed: Sep. 29, 2023

(51) Int. Cl. A61M 5/32; A61M 5/34

(52) U.S. Cl. CPC A61M 5/326; A61M 5/3202; A61M 5/34

(57) ABSTRACT

An injection system including a housing having a first end and a second end opposite the first end, the housing defining an interior space, a needle having an unshielded state and a shielded state, the needle having a first end and a second end positioned opposite the first end, the second end having a piercing tip, at least a portion of the needle is positioned within the housing, and a syringe adapter connection interface configured to mate with a corresponding connection interface of a syringe adapter, with the syringe adapter connection interface having a membrane and defining a passageway.

### BACKGROUND OF THE INVENTION

### Field of the Disclosure

The present disclosure relates generally to an injection system and, more specifically, an injection system for use with a closed system transfer device.

### Description of the Related Art

Health care providers reconstituting, transporting, and administering hazardous drugs, such as cancer treatments, can put health care providers at risk of exposure to these medications and present a major hazard in the health care environment. For example, nurses treating cancer patients risk being exposed to chemotherapy drugs and their toxic effects. Unintentional chemotherapy exposure can affect the nervous system, impair the reproductive system, and bring an increased risk of developing blood cancers in the future. In order to reduce the risk of health care providers being exposed to toxic drugs, the closed transfer of these drugs becomes important.

Some drugs must be dissolved or diluted before they are administered, which involves transferring a solvent from one container to a sealed vial containing the drug in powder or liquid form using a needle. Drugs may be inadvertently released into the atmosphere in gas form or by way of aerosolization, during the withdrawal of the needle from the vial and while the needle is inside the vial if any pressure differential between the interior of the vial and the surrounding atmosphere exists. In order to reduce the risk of health care providers being exposed to toxic drugs, the transfer of these drugs is accomplished utilizing a closed system transfer device or system. A syringe adapter and vial adapter are utilized to transfer medication between a syringe and a vial. A patient connector, which can be connected to an infusion line, may be connected to the syringe adapter to delivery medication via the infusion line. In instances where direct injection into a patient is required, a needle is connected to the patient connector.

### SUMMARY OF THE INVENTION

In one aspect or embodiment of the present application, the injection system includes a housing having a first end and a second end opposite the first end, the housing defining an interior space, a needle having an unshielded state and a shielded state, the needle having a first end and a second end positioned opposite the first end, the second end having a piercing tip, at least a portion of the needle is positioned within the housing, and a syringe adapter connection interface configured to mate with a corresponding connection interface of a syringe adapter, the syringe adapter connection interface having a membrane and defining a passageway.

In a further aspect or embodiment, when the needle is in the unshielded state, the needle has a first position with the piercing tip of the needle positioned outside of the housing, where, when the needle is in the shielded state, the needle has a second position with the piercing tip of the needle positioned within the interior space of the housing, and where the needle is moveable between the first position and the second position.

In another aspect or embodiment, the injection system includes a needle shield, where when the needle is in the unshielded state, the needle shield has a first position with the piercing tip of the needle positioned outside of the needle shield, where, when the needle is in the shielded state, the needle shield has a second position with the piercing tip of the needle positioned within the needle shield, and wherein the needle shield is moveable between the first position and the second position.

In one aspect or embodiment, the membrane seals the passageway within the syringe adapter connection interface. In a further aspect or embodiment, the injection system includes a saline solution positioned within a portion of the housing or a portion of the syringe adapter connection interface. In another aspect, the needle and the passageway of the syringe adapter connection interface are in fluid communication.

In a further aspect or embodiment, the injection system includes a needle sleeve having a first position where the needle sleeve encloses the needle and a second position where the piercing tip of the needle is positioned outside of the needle sleeve. In another aspect or embodiment, the injection system includes a needle cover engaged with the housing and covering the piercing tip of the needle, the needle cover configured to be removed from the housing.

In one aspect or embodiment, the syringe adapter connection interface of the injection system includes an interface body having a first end and a second end, and wherein the housing further includes a housing luer connection, the housing luer connection configured to mate with an interface luer connection of the interface body. In a further aspect or embodiment, a syringe adapter is configured to receive the syringe adapter connection interface such that the syringe adapter receives the membrane of the syringe adapter connection interface.

In one aspect or embodiment, a method of using the injection system of any of above aspects or embodiments includes: filling a portion of the housing or a portion of the syringe adapter connection interface with saline solution; and engaging the syringe adapter connection interface with the housing.

In a further aspect or embodiment, the method includes moving the needle from the shielded state to the unshielded state. In another aspect or embodiment, moving the needle from the shielded state to the unshielded state includes moving a needle shield from the second position to the first position when the needle is in the first position. In another aspect or embodiment, moving the needle from the shielded state to the unshielded state includes moving the needle from the first position to the second position. In a further aspect or embodiment, the method includes inserting the syringe adapter connection interface into the syringe adapter such that the membrane is received within the syringe adapter.

### BRIEF DESCRIPTION OF THE DRAWINGS

The above-mentioned and other features and advantages of this disclosure, and the manner of attaining them, will become more apparent and the disclosure itself will be better understood by reference to the following descriptions of embodiments of the disclosure taken in conjunction with the accompanying drawings, wherein:

FIG. 1 is a perspective view of an injection system according to one aspect or embodiment of the present application.

FIG. 2A is a front view of the injection system of FIG. 1.

FIG. 2B is a cross-sectional view taken along line A-A shown in FIG. 2A.

FIG. 3 is a bottom view of the injection system of FIG. 1.

FIG. 4 is a front view of a portion of an injection system according to a further aspect or embodiment of the present application, showing an unshielded position of a needle.

FIG. 5 is a front view of the portion of the injection system of FIG. 4, showing a shielded position of a needle.

FIG. 6 is a perspective view of the portion of the injection system of FIG. 4, showing a shielded position of a needle.

FIG. 7A is a cross-sectional view of a conventional syringe adapter and patient connector.

FIG. 7B is a front view of the syringe adapter and patient connector of FIG. 7A.

### DETAILED DESCRIPTION

The following description is provided to enable those skilled in the art to make and use the described embodiments contemplated for carrying out the invention. Various modifications, equivalents, variations, and alternatives, however, will remain readily apparent to those skilled in the art. Any and all such modifications, variations, equivalents, and alternatives are intended to fall within the spirit and scope of the present invention.

For purposes of the description hereinafter, the terms “upper”, “lower”, “right”, “left”, “vertical”, “horizontal”, “top”, “bottom”, “lateral”, “longitudinal”, and derivatives thereof shall relate to the invention as it is oriented in the drawing figures. However, it is to be understood that the invention may assume various alternative variations, except where expressly specified to the contrary. It is also to be understood that the specific devices illustrated in the attached drawings, and described in the following specification, are simply exemplary embodiments of the invention. Hence, specific dimensions and other physical characteristics related to the embodiments disclosed herein are not to be considered as limiting.

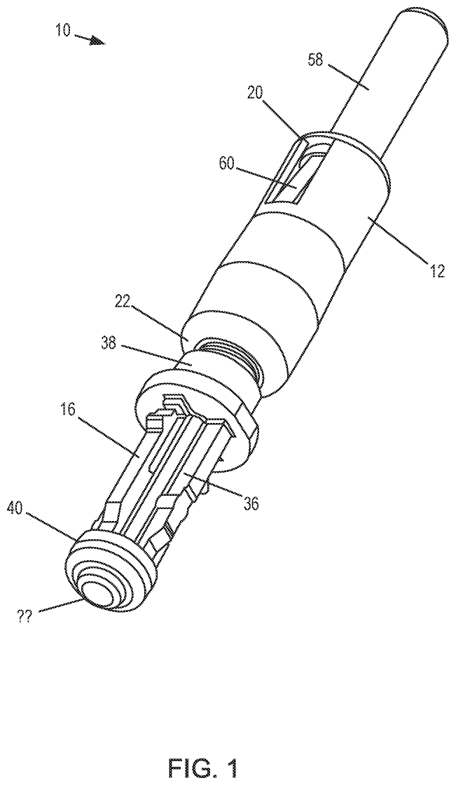

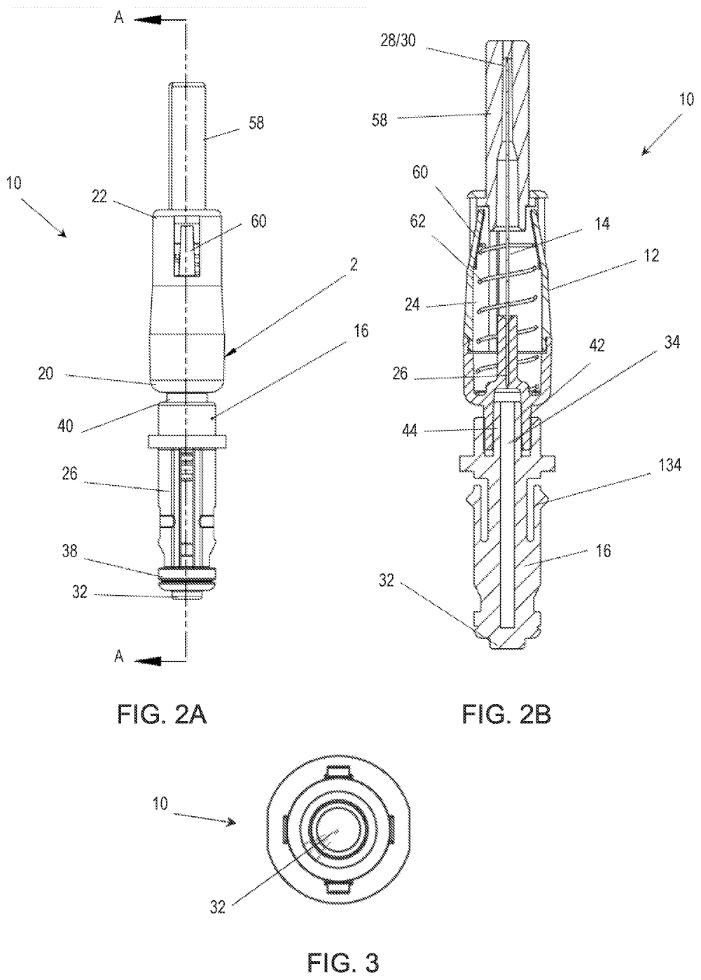

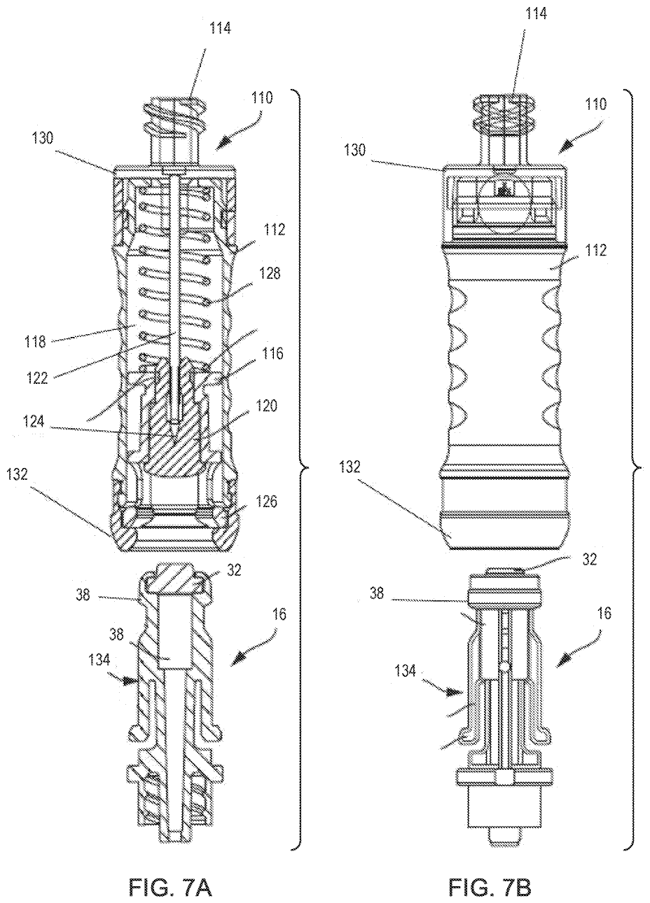

Referring to FIGS. 1-3, 7A, and 7B, an injection system 10 according to one aspect or embodiment includes a housing 12, a needle 14, and a syringe adapter connection interface 16. The injection system 10 may form part of a closed system transfer device or system utilized to facilitate the closed transfer of fluids between containers and to facilitate the administration of medication to a patient without exposing the ambient environment to the medication. The injection system is configured to cooperate with other components of a closed system transfer device or system, such as a syringe adapter, to allow medication to be directly injected from a syringe into a patient while ensuring safe administration and preventing exposure of the medication to the ambient environment. The syringe adapter connection interface 16 is configured to be received by a syringe adapter 18, such as the syringe adapter 18 shown in FIGS. 7A and 7B, as described in U.S. Patent Application No. 2018/0200148, which is incorporated by reference in its entirety. The syringe adapter connection interface 16, however, may be configured to connect with other suitable syringe adapters.

Referring to FIGS. 1-3, the housing 12 includes a first end 20 and a second end 22 positioned opposite the first end 20 and defines an interior space 24. The needle 14 has an unshielded state and a shielded state (shown in FIGS. 1-3). The needle 14 has a first end 26 and a second end 28 positioned opposite the first end 26. The second end 28 of the needle 14 has a piercing tip 30. The piercing tip 30 is configured to pierce a patient's skin to provide a subcutaneous injection of medication. At least a portion of the needle 14 is positioned within the housing 12. The syringe adapter connection interface 16 is configured to mate with a corresponding connection interface of the syringe adapter 18, as discussed in more detail below. The syringe adapter connection interface 16 includes a membrane 32 and defines a passageway 34. In one embodiment, the membrane 32 seals the passageway 34 within the syringe adapter connection interface 16. In a further aspect or embodiment, a saline solution (not shown) is positioned within a portion of the housing 12 or a portion of the syringe adapter connection interface 16, which allows the injection system 10 and the needle 14 to be pre-primed. The syringe adapter connection interface 16 may be the same or similar to the syringe adapter connection interface shown and described in U.S. Patent Application No. 2018/0200148.

Still referring to FIGS. 1-3, the syringe adapter connection interface 16 includes an interface body 36 having a first end 38 and a second end 40 positioned opposite to the first end 38. The housing 12 includes a housing luer connection 42, with the housing luer connection 42 configured to mate with an interface luer connection 44 of the interface body 36. When the syringe adapter connection interface 16 and the housing 12 are connected together, the passageway 34 of the syringe adapter connection interface 16 and the needle 14 are in fluid communication. In this manner, when the housing 12 and the syringe adapter connection interface 16 are connected together, for example by inserting the second end 22 of the housing 12 into interface body 36 of the syringe adapter connection interface 16, the needle 14 is pre-primed with a solution present in a portion of the housing 12 and/or the passageway 34 of the syringe adapter connection interface 16. A solution such as a saline solution can be positioned in a portion of the housing 12 and/or the passageway 34 of the syringe adapter connection interface 16 before connecting the syringe adapter connection interface 16 and the housing 12 together. In one aspect or embodiment, a portion of the housing 12 and/or the passageway 34 can be pre-filled prior to packaging the injection system 10. In this manner, when the syringe adapter connection interface 16 is connected to the housing 12, the saline solution is exposed to the ambient environment and not the medication. In another aspect or embodiment, rather than forming the syringe adapter connection interface 16 and the housing 12 separately and connecting them together, the housing 12 and the syringe adapter connection interface 16 may be formed integrally.

Referring again to FIGS. 1-3, the injection system 10 further includes a needle shield 58. When the needle 14 is in the unshielded state, the needle shield 58 has a first position with the piercing tip 30 of the needle 14 position outside of the needle shield 58. When the needle 14 is in the shielded state, the needle shield 58 has a second position with the piercing tip 30 of the needle 14 positioned within the needle shield 58. The needle shield 58 is movable between the first position and the second position. In one aspect or embodiment, the needle shield 58 is at least partially positioned within the housing 12 in the first position and extends outside of the housing 12 to cover the piercing tip 30 of the needle 14 in the second position. The needle shield 58 may be actuated from the first position to the second position by depressing a button 60, which disengages the needle shield 58 from the housing 12 and allows a spring 62 to bias the needle shield 58 to the second position. In a further aspect or embodiment, the needle shield 58 is biased to the second position with the needle 14 in the shielded state and depressing the needle shield 58 against the skin of a patient causes the needle shield 58 to retract to the first position to allow for a subcutaneous injection and subsequently returns to the second position upon removal of the needle 14 from the patient. In one aspect, the needle shield 58 prevents exposure of the medication to the ambient environment. The needle shield 58, housing 12, and/or other component may include a plug or other structure that seals the piercing tip 30 of the needle 14 when the needle 14 is in the shielded state.

In one aspect or embodiment, the injection system 10 may include a needle sleeve (not shown) having a first position where the needle sleeve encloses the needle 14 and a second position where the piercing tip 30 of the needle 14 is positioned outside of the needle sleeve. The needle sleeve may be moved from the first position to the second position during use of the needle 14. The needle sleeve may be formed from an elastomeric material. When the piercing tip 30 of the needle 14 enters the skin of a patient the needle sleeve is compressed or retracted, which exposes the piercing tip 30 of the needle 14 to be inserted into a patient. Upon completion of the injection, the needle sleeve returns to the first position when the needle sleeve decompresses into its original shape to enclose the needle 14. The needle sleeve is configured to prevent any leakage from the needle 14 to ensure the medication is not exposed to the ambient environment.

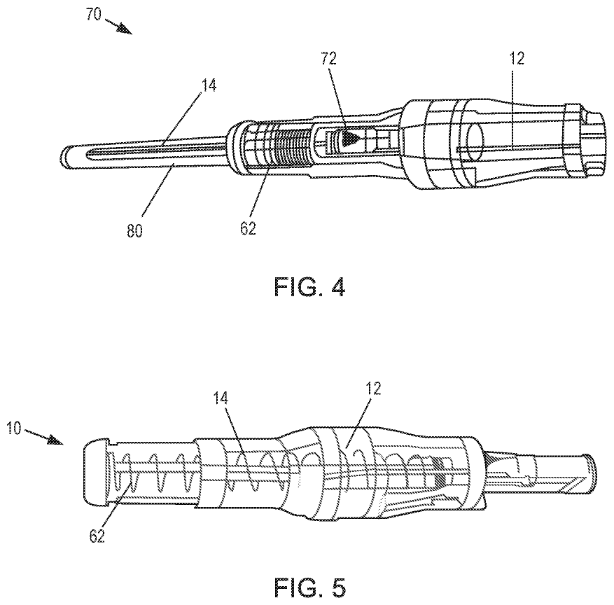

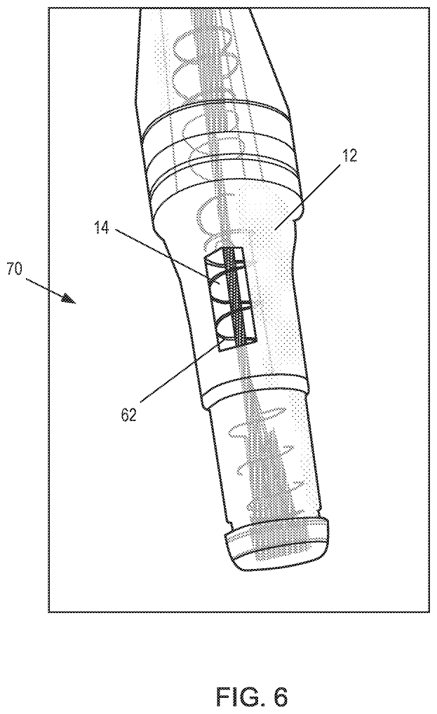

Referring to FIGS. 4-6, an injection system 70 according to a further aspect or embodiment is shown. The injection system 70 of FIGS. 4-6 is similar to the injection system 10 of FIGS. 1-3, except for the differences discussed below. In the injection system 70 of FIGS. 4-6, when the needle 14 is in the unshielded state, the needle 14 has a first position with the piercing tip 30 of the needle 14 positioned outside of the housing 12. In the shielded state, the needle 14 has a second position with the piercing tip 30 of the needle 14 positioned within the interior space 24 of the housing 12. The needle 14 is moveable between the first position and the second position. The needle 14 may be moved, for example, by actuating a button 72 to disengage a needle hub holding the needle 14 from the housing 12 to allow a spring 74 to bias the needle hub and needle 14 within the housing 12. In this manner, the needle 14 is moved from the first position to the second position after completing an injection in order to prevent needle stick injuries and/or seal the piercing tip 30 of the needle 14 to prevent exposure of the medication to the ambient environment. In one aspect or embodiment, the arrangement for retracting the needle 14 within the housing may be the same or similar to the BD Vacutainer® push button blood collection set commercially available from Becton, Dickinson and Company.

Referring to FIG. 4, in one aspect or embodiment, the injection system 70 as well as the injection system 10 of FIGS. 1-3 may further include a needle cover 80. The needle cover 80 is engaged with the housing 12 and covers the piercing tip 30 of the needle 14. The needle cover 80 is configured to be removed from the housing 12 prior to use of the injection system 10, 70.

Referring to FIGS. 7A and 7B, in one aspect or embodiment, the injection system 10, 70 includes the syringe adapter 18, which is configured to receive the syringe adapter connection interface 16. The syringe adapter 18 is one component of a system for the closed transfer of fluids. In particular, the syringe adapter 18 is configured to connect to a syringe (not shown) and to another medical device or fluid container, such as the injection system 10, 70. The medical device can be, for example, a patient line, vial adapter, fluid container, or infusion adapter. The container can be a medical vial, syringe barrel, IV bag, or similar container for holding a fluid to be administered to a patient. The syringe adapter 18 can be used to facilitate the closed transfer of fluids between the syringe and medical device or fluid container. The syringe adapter is shown and described in U.S. Patent Application Publication No. 2018/0200147.

Referring again to FIGS. 7A and 7B, the syringe adapter 18 includes an outer housing 112, a syringe connection 114, and a membrane housing 116. The syringe connection 114 is configured to be connected to a syringe. The syringe connection 114 may be a luer connector, although other suitable connection arrangements may be utilized. The membrane housing 116 is positioned within an interior space 118 of the outer housing 112 and is moveable from a first position within the outer housing 112 to a second position within the outer housing 112 that is spaced from the first position. The membrane housing 116 receives a membrane 120. The syringe adapter 18 further includes a needle 122 received within the interior space 118 of the outer housing 112. A piercing tip 124 of the needle 122 is positioned within the membrane 120 when the membrane housing 116 is in the first position and the piercing tip 124 of the needle 122 is positioned outside of the membrane 120 when the membrane housing 116 is in the second position. The membrane housing 116 includes a collet 126 configured to receive the syringe adapter connection interface 16 of the injection system 10, 70. The syringe adapter 18 also includes a spring 128 received within the interior space 118 of the outer housing 112. The spring 128 biases the membrane housing 116 toward the first position. The outer housing 112 has a first end 130 and a second end 132 positioned opposite the first end 130.

When the syringe adapter connection interface 16 is inserted into the outer housing 112 of the syringe adapter 18, the collet 126 of the membrane housing 116 receives the syringe adapter connection interface 16 and moves the membrane housing 116 within the outer housing 112 toward the first end 130 of the outer housing 112. As the membrane housing 116 moves, the collet 126 secures the membrane housing 116 to the syringe adapter connection interface 16 to ensure engagement between the membrane 32 of the syringe adapter connection interface 16 and the membrane 120 of the membrane housing 116. As the membrane housing 116 is moved from the first position to the second position, the piercing tip 124 of the needle 122 pierces the membrane 120 of the membrane housing 116 and the membrane 32 of the syringe adapter connection interface 16 such that the piercing tip 124 is received within the passageway 34 thereby placing the needle 122 in fluid communication with the syringe adapter connection interface 16. The syringe connection 114 is in fluid communication with the needle 122 to allow a syringe (not shown) connected to the syringe connection 114 to be in fluid communication with the syringe adapter connection interface 16 when the syringe adapter 18 is connected to the syringe adapter connection interface 16. Moving the membrane housing 116 from the second position to the first position, which is aided by the spring 128, releases the syringe adapter connection interface 16 from the collet 126 to separate the syringe adapter connection interface 16 from the syringe adapter 18. The membrane housing 116 is secured in the second position by a locking arrangement 134 of the syringe adapter connection interface 16 that engages a portion of the outer housing 112 to hold the membrane housing 116 in the second position. Pulling the syringe adapter connection interface 16 away from the syringe adapter 18 releases the locking arrangement 134 to allow the membrane housing 116 to move back to the first position from the second position.

When the syringe adapter 18 is connected to the syringe adapter connection interface 16, which is connected to the housing 12, the medication within the syringe connected to the syringe connection 114 of the syringe adapter 18 is not exposed to the ambient environment due to pre-primed needle 14 and/or sealing the needle 14 of the injection device 10, 70. As noted above, other suitable syringe adapters may also be utilized in connection with the injections system.

In a further aspect or embodiment, a method of using the injection system 10, 70 includes filling the passageway 34 of the syringe adapter connection interface 16 or a portion of the housing 12 with saline solution, and engaging the syringe adapter connection interface 16 with the housing 12. The method may include pre-priming the needle 14 by attaching a syringe to the syringe adapter connection interface 16 and depressing a plunger of the syringe to push the saline through the needle 14. The method may include attaching a syringe with a medication to the syringe adapter 18 and connecting the syringe adapter 18 to the syringe adapter connection interface 16, injecting the medication through the needle 14 of the injection system 10, 70 into a patient while the needle 14 is in the unshielded position, withdrawing the needle 14 from the patient and placing the needle 14 in the shielded position.

While this disclosure has been described as having exemplary designs, the present disclosure can be further modified within the spirit and scope of this disclosure. This application is therefore intended to cover any variations, uses, or adaptations of the disclosure using its general principles. Further, this application is intended to cover such departures from the present disclosure as come within known or customary practice in the art to which this disclosure pertains and which fall within the limits of the appended claims. To the extent possible, one or more features of any aspect or embodiment discussed above can be combined with one or more features of any other aspect or embodiment.

1. An injection system comprising:
a housing having a first end and a second end opposite the first end, the housing defining an interior space;
a needle having an unshielded state and a shielded state, the needle having a first end and a second end positioned opposite the first end, the second end having a piercing tip, at least a portion of the needle is positioned within the housing; and
a syringe adapter connection interface configured to mate with a corresponding connection interface of a syringe adapter, the syringe adapter connection interface comprising a membrane and defining a passageway.

2. The injection system of claim 1, wherein when the needle is in the unshielded state, the needle has a first position with the piercing tip of the needle positioned outside of the housing, wherein, when the needle is in the shielded state, the needle has a second position with the piercing tip of the needle positioned within the interior space of the housing, and wherein the needle is moveable between the first position and the second position.

3. The injection system of claim 1, further comprising a needle shield, wherein when the needle is in the unshielded state, the needle shield has a first position with the piercing tip of the needle positioned outside of the needle shield, wherein, when the needle is in the shielded state, the needle shield has a second position with the piercing tip of the needle positioned within the needle shield, and wherein the needle shield is moveable between the first position and the second position.

4. The injection system of claim 1, wherein the membrane seals the passageway within the syringe adapter connection interface.

5. The injection system of claim 4, further comprising a saline solution positioned within a portion of the housing or within a portion of the syringe adapter connection interface.

6. The injection system of claim 1, wherein the needle and the passageway of the syringe adapter connection interface are in fluid communication.

7. The injection system of claim 1, further comprising a needle sleeve having a first position where the needle sleeve encloses the needle and a second position where the piercing tip of the needle is positioned outside of the needle sleeve.

8. The injection system of claim 1, further comprising a needle cover engaged with the housing and covering the piercing tip of the needle, the needle cover configured to be removed from the housing.

9. The injection system of claim 1, wherein the syringe adapter connection interface comprises an interface body having a first end and a second end, and wherein the housing further comprises a housing luer connection, the housing luer connection configured to mate with an interface luer connection of the interface body.

10. The injection system of claim 1, further comprising a syringe adapter configured to receive the syringe adapter connection interface, wherein the syringe adapter receives the membrane of the syringe adapter connection interface.

11. A method of using the injection system of claim 1, the method comprising:
filling a portion of the housing or a portion of the syringe adapter connection interface with saline solution; and
engaging the syringe adapter connection interface with the housing.

12. The method of claim 11, further comprising:
moving the needle from the shielded state to the unshielded state.

13. The method of claim 12, wherein moving the needle from the shielded state to the unshielded state comprises moving a needle shield from the second position to the first position when the needle is in the first position.

14. The method of claim 12, wherein moving the needle from the shielded state to the unshielded state comprises moving the needle from the first position to the second position.

15. The method of claim 11, further comprising inserting the syringe adapter connection interface into the syringe adapter such that the membrane is received within the syringe adapter.
